(19) United States
(12) Patent Application Publication
(10) Pub. No.: US 2012/0039831 A1
(43) Pub. Date: Feb. 16, 2012

(54) Natural skin and lip colorant compositions

(76) Inventor: Stephen Vincent Musumeci, Cresco, PA (US)

(21) Appl. No.: 13/136,757

(22) Filed: Aug. 10, 2011

Related U.S. Application Data
(60) Provisional application No. 61/401,254, filed on Aug. 10, 2010.

(51) Int. Cl. A61K 8/97; A61Q 1/04; A61Q 1/02; A61Q 1/06

(52) U.S. Cl. 424/64; 424/63

(57) ABSTRACT

Skin or lip colorant compositions include a dispersion of a colored herbal extract powder in a vegetable oil base and can contain various other ingredients such as a natural colorant mineral ore powder, a natural or naturally derived thickening agent, a vegetable based anhydrous wax base, lanolin or a lanolin derivative. The colorant compositions can also be provided as powdered compositions by absorbing the ingredients onto natural dry carrier ingredients. Other optional ingredients can include surfactants, emollients, moisturizing agents, absorbent powders, vitamins, minerals, flavors and fragrances. Preferably the skin or lip colorant composition is free of petroleum based ingredients as well as synthetically derived emollients and moisturizers.

### REFERENCE TO RELATED APPLICATIONS

This application claims the benefit of provisional application Ser. No. 61/401,254 filed Aug. 10, 2010.

### FIELD OF INVENTION

The present invention relates to natural or naturally derived cosmetic compositions for coloring the skin and lips.

### BACKGROUND OF THE INVENTION

There are a large variety of skin complexion formulations, colorant, lipsticks, lip glosses and similar cosmetic products, which are marketed for modifying skin color, lip color, gloss or appearance. Such cosmetic compositions use a broad variety of colorants most of which are synthetic.

Many of these colorants may be absorbed through the skin and may be metabolized into toxins by the human body. Some colorants may be sensitizers due to absorption and re-emission of UV light from the sun. There is increasing recognition of the desirability of using natural ingredients, which are metabolized into safe degradation products by the body. Carminic acid, a safer alternative to synthetic dyes, is widely used as a red colorant especially in lipsticks. While natural, this colorant is derived from the dried bodies of the cochineal insect. Many consumers do not like the concept of consuming such a dye. Vegetarians and people adhering to a kosher or halal diet may also find the use of such a dye as unacceptable. It would seem that there is a need for cosmetic products using natural or naturally derived ingredients which use dyes from herbal sources. Accordingly this application is directed towards the provision of safe natural herbal cosmetics for skin and lip coloration.

### SUMMARY OF THE INVENTION

The present invention is a skin or lip colorant composition comprising a dispersion of a colored herbal extract powder in a vegetable oil base and containing various other ingredients such as a natural colorant mineral ore powder, a natural or naturally derived thickening agent, a vegetable based anhydrous wax base, lanolin or a lanolin derivative. The colorant composition can also be provided as a powdered composition by absorbing the ingredients onto natural dry carrier ingredients. Other optional ingredients may be included in the composition such as surfactants, emollients, moisturizing agents, absorbent powders, vitamins, minerals, flavors and fragrances, etc. Preferably the skin or lip colorant composition is free of petroleum based ingredients as well as synthetically derived emollients and moisturizers.

### DETAILED DESCRIPTION

The present invention is a skin or lip colorant composition comprising a dispersion of from about 1% to about 15 wt. % micronized colored herbal extract powder in from about 4% to about 60 wt. % of a natural or naturally derived vegetable oil and one of more of the following (1) from about 0.5% to about 6 wt. % a natural colorant mineral ore powder, (2) from 1% to about 10 wt. % of a natural or naturally derived thickening agent, (3) from about 2% to about 50 wt. % of a natural or naturally derived emollient liquid ester of a C₁ to a C₁₈ alcohol with a C₁₂ to a C₁₈ fatty acid, (4) from about 1% to about 50 wt. % of a natural or naturally derived ester of a C₁ to a C₁₈ alcohol with a poly-carboxylic acid, (5) from about 5% to about 20 wt. % of a vegetable based anhydrous wax, (6) from about 1% to about 15 wt. % lanolin or lanolin derivative or (7) from about 20% to about 80 wt. % of a natural or naturally derived powder base. Other ingredients may be included in the composition if desired including but not limited to surfactants, antioxidants, preservatives, fragrances, flavors, sun screens and processing aids etc. However, the skin or lip composition is essentially free of petroleum ingredients and synthetically based emollients or moisturizers.

This invention also discloses a method of coloring skin or lip by applying a composition comprising a dispersion of from about 1% to about 15 wt. % micronized colored herbal extract powder dispersed in from about 4% to about 60 wt. % of a natural or naturally derived vegetable oil and further comprising optionally one of more of the following: (1) from about 0.5% to about 6 wt. % a natural colorant mineral ore powder, (2) from 1% to about 10 wt. % of a natural or naturally derived thickening agent, (3) from about 2% to about 50 wt. % of a natural or naturally derived emollient liquid ester of a C₁ to a C₁₈ alcohol and a C₁₂ to a C₁₈ fatty acid, (4) from about 1% to about 50 wt. % of a natural or naturally derived ester of a C₁ to a C₁₈ alcohol with a poly-carboxylic acid, (5) from about 5% to about 20 wt. % of a vegetable based anhydrous wax, (6) from about 1% to about 15 wt. % lanolin or lanolin derivative or (7) from about 20% to about 80 wt. % of a natural powder base.

Micronized herbal extract colorant powders are used at concentrations between about 1 and 15 wt. %. For a lipstick it is preferable to incorporate from about 4% to about 10 wt. % herbal extract colorant. For an eye shadow between about 6 to about 15 wt. % of herbal extract colorant is needed. For a cosmetics where slight modification of skin color is desired such as for a light facial product, from about 1 to about 4 wt. % herbal extract colorant is sufficient. The choice of herbal extract used depends on the color desired. Many sources of herbal extract powders can be used in compositions of this invention. One group of natural colored dyes suitable for lipsticks, foundation creams, skin colorants etc are marketed under the trademark Microzest (Lessonia, Saint-Thonan, Bretagne, France). These include but are not limited to red shades, such as quinoa, rose, carrot pink grind and anthocyan pink grind, brown shades such as argan, lychee, coconut and apple, yellow shades such as carrot orange grind, bitter orange and lemon, blue shades such as indigo and anthocyan, gardenia blue grind, and green shades such as green tea.

Another group of useful hydrophobic red herbal extracts are the naphthazarin derivatives which are extracted from Boraginaceae such as alkanet. Another useful red dye is annatto which is extracted from achiote trees. The Madder Lake is another red herbal extract that has been made into an insoluble lake using aluminum. A purified ingredient from rose madder is alizarin red pigment. Another useful natural deep red dye is Hopi Red Dye. Beet juice powder is another useful red colorant.

Other colors are available including a pigment called gamboge, which is a yellow extract from trees. Various carotenoids, which are yellow or brownish, are also useful dyes. Carotenoids, such as beta-carotene, are found in many fruits and vegetables including beats, tomatoes and carrots. Henna is another useful natural dye with various shades of yellow and brown. Other yellow vegetable dyes include turmeric and saffron. A useful green dye is chlorophyll.

Herbal extract colorants used in this invention are generally somewhat hydrophobic, which makes them dispersible in various oils. Natural vegetable oils are an ideal medium for making this suspension. In addition vegetable oils can increase the gloss on lips. One of the best vegetable oils for lipsticks, lip glosses, lip colorant liquids and lip colorant lotions is castor oil. Castor oil also acts as a plasticizer when used with waxes. However, many other oils of vegetable origin can be used. These include but are not limited to corn oil, cottonseed oil, palm oil, safflower oil, olive oil, rapeseed oil, peanut oil, soybean oil as well as vegetable-sourced squalene and squalane. Naturally derived vegetable oils in which a natural fat or oil has been slightly modified are also useful in compositions of this invention. For examples the oils can be partially or fully hydrogenated.

Since the herbal extract colorant powders provide relatively mild coloration, from about 0.5% to 6 wt. % natural colorant minerals can be added to the herbal extract colorants to enhance the color. Natural colorant minerals include but are not limited to inorganic pigments such as red ochre and yellow ochre iron oxides, sienna limonite clay, umber clay, zinc oxide, titanium dioxide, ultramarine blue from lapis lazuli.

Natural or naturally derived thickening agents for use in lipsticks and glosses include but are not limited to natural fatty acids with from about 14 to about 24 carbon atoms and the alkali metal salts of fatty acids with about 12 to about 18 carbon atoms. Some oil soluble esters of fatty acids with from about 14 to about 24 carbon atoms, such as palm butter, are also useful as thickening agents in formulations of the invention. Water soluble or dispersible sucrose esters of aliphatic acids and 12-hydroxy stearic acid can also be used.

For liquid and stick products like lipstick and lip gloss, it is desirable to add liquid emollients and moisturizers in addition to the vegetable oils. These include esters such as C₁ to C₁₈ alcohol esters of C₁₂ to C₂₄ fatty acids with a melting points below room temperature, for example 20° C., are useful. Non-limiting examples of suitably esters are the methyl, ethyl, propyl, isopropyl, butyl, isobutyl and ethyl hexyl, decyl, lauryl, myristyl, palmityl and cetyl esters of lauric, myristic, palmitic, oleic and stearic acids. Examples are isopropyl isostearate, octyldodecyl erucate (ester of erucic acid), octyl oxystearate, cetyl octanoate, octyldodecyl oleate, oleyl oleate, isocetyl stearate, isopropyl palmitate, octyldodecyl myristate, myristyl myristate, decyl oleate, and octyldodecyl ricinoleate

Also useful as emollients are the esters of C₈ to C₁₈ alcohols with polycarboxylic acids. For example, monohydric alcohol esters of adipic acid, succinic acid or malic acid, tartaric acid, citric acid, malonic acid can be used. Examples are diisostearyl adipate, dioctyl adipate, dioctyl succinate, diisostearyl malate, trioctyldodecyl citrate.

Waxes are an important component in solid lipsticks but may even be included at lower levels in lip glosses, and lip colorant liquids and lotions. Vegetable waxes may be soft or hard depending on the desired consistency of the final product. Useful waxes for lipsticks of the invention include but are not limited to beeswax, candelilla wax, sunflower wax, carnauba wax, rice bran wax, orange peel wax, grapefruit wax, lemon peel wax, sunflower wax and sumac wax. Powders for coloring the skin can utilize the same type of natural dye suspended in a natural oil. However, for skin colorant powder a carrier powder onto which the oil suspension can be absorbed is needed. There are many suitable natural or naturally derived powders for this use including but not limited to talc, corn starch, microcrystalline cellulose, micronized calcium carbonate, kaolin, mica, bentonite clay etc.

Skin and lip colorants of the invention can be used for many different cosmetic uses. For example, compositions of the invention can be used as a skin blush, eye shadow, blemish cover, lipstick, lip colorant lotion etc.

All measures are in weight unless otherwise specified.

The invention may be further illustrated by way of the following examples:

### Example 1

| Natural Lipstick | Wt. % |
|---|---|
| Sunflower | 7.80 |
| Rice bran wax | 3.90 |
| Carnauba wax | 1.60 |
| Grapefruit wax | 0.85 |
| Orange peel wax | 0.85 |
| Lemon peel wax | 1.75 |
| Ethyl hexyl palmitate | 14.05 |
| Palm butter | 1.95 |
| Trooctyldodecyl citrate | 16.00 |
| Castor Oil | 11.35 |
| 30% Zinc oxide in castor oil | 4.56 |
| 50% Titanium dioxide in castor oil | 4.56 |
| 30% Iron oxide gring in castor oil | 5.70 |
| 25% Microzest 25 carrot orange grind in castor oil | 4.56 |
| 25% Microzest 25 carrot pink grind in castor oil | 4.56 |
| 25% Microzest 25 gardenia blue grind in castor oil | 5.70 |
| 25.% Microzest 25 anthocyan pink grind in castor oil | 4.56 |
| 25% Microzest 25 gardenia blue grind in castor oil | 5.70 |

1. A skin or lip colorant composition comprising a dispersion of from about 1% to about 15 wt. % micronized colored herbal extract powder in from about 4% to about 60 wt. % of a natural or naturally derived vegetable oil and further comprising optionally one of more of the following: (1) from about 0.5% to about 6 wt. % a natural colorant mineral ore powder, (2) from 1% to about 10 wt. % of a natural or naturally derived thickening agent, (3) from about 2% to about 50 wt. % of a natural or naturally derived emollient liquid ester of a C₁ to a C₁₈ alcohol and a C₁₂ to a C₁₈ fatty acid, (4) from about 1% to about 50 wt. % of a natural or naturally derived ester of a C₁ to a C₁₈ alcohol with a poly-carboxylic acid, (5) from about 5% to about 20 wt. % of a vegetable based anhydrous wax, (6) from about 1% to about 15 wt. % lanolin or lanolin derivative or (7) from about 20% to about 80 wt. % of a natural powder base.

2. A skin or lip colorant of claim 1 in which the micronized colored herbal extract powder is selected from the group consisting of quinoa, rose, carrot pink, anthocyan pink, argan, lychee, coconut apple, carrot orange, bitter orange, lemon, indigo, gardenia blue and green tea.

3. A skin or lip colorant of claim 1 in which the micronized colored herbal extract powder comprises a naphthazarin derivative that is extracted from a complex selected from the group consisting of Boraginaceae, annatto, madder lake, alizarin red pigment. Hopi red dye, and beet juice powder.

4. A skin or lip colorant of claim 1 in which the micronized colored herbal extract powder is selected from the group consisting of gamboge, carotenoids, beta-carotene, henna turmeric, saffron and chlorophyll.

5. A skin or lip colorant of claim 1 in which the colored herbal extract powder is dispersed in a natural or naturally derived vegetable oil selected from the group consisting of castor oil, corn oil, cottonseed oil, palm oil, safflower oil, olive oil, rapeseed oil, peanut oil, soybean oil, vegetable-sourced squalene and squalane.

6. A skin or lip colorant of claim 1 in which the colored herbal extract powder is dispersed in a natural or naturally derived vegetable oil that has been partially or fully hydrogenated.

7. A skin or lip colorant of claim 1; which contains from about 0.5 to about 6 wt. % of a natural colorant mineral ore pigment selected from the group consisting of red ochre iron oxide, yellow ochre-iron oxides, sienna limonite clay, umber clay, zinc oxide, titanium dioxide, Lapis lazuli ultramarine blue.

8. A skin or lip colorant of claim 1, which contains from 1% to about 10 wt. % of a natural or naturally derived thickening agent selected from the group consisting of natural fatty acids with from about 14 to about 24 carbon atoms, alkali metal salts of fatty acids with about 12 to about 18 carbon atoms, palm butter, and water soluble or dispersible sucrose esters of aliphatic acids.

9. A skin or lip colorant of claim 1 which contains emollient esters such as C₁ to C₁₈ alcohol esters of C₁₂ to C₂₄ fatty acids with melting points below 20° C.

10. A skin or lip colorant of claim 9 containing an emollient esters selected from the group consisting of methyl, ethyl, propyl, isopropyl, butyl, isobutyl and ethyl hexyl, decyl, lauryl, myristyl, palmityl and cetyl esters of lauric, myristic, palmitic, oleic and stearic acids.

11. A skin or lip colorant of claim 9 containing an emollient esters selected from the group consisting of isopropyl isostearate, octyldodecyl erucate (ester of erucic acid), octyl oxystearate, cetyl octanoate, octyldodecyl oleate, oleyl oleate, isocetyl stearate, isopropyl palmitate, octyldodecyl myristate, myristyl myristate, decyl oleate, and octyldodecyl ricinoleate

12. A skin or lip colorant of claim 9 containing emollient esters selected from the group consisting of esters of C₈ to C₁₈ alcohols with polycarboxylic acids.

13. A skin or lip colorant of claim 12 in which the emollient ester is a monohydric alcohol ester of an acid selected from the group consisting of adipic acid, succinic acid, malic acid, tartaric acid, citric acid, and malonic acid.

14. A skin or lip colorant of claim 1 which contains from about 5% to about 20 wt. % of a vegetable wax

15. A skin or lip colorant of claim 14 in which the wax is selected from the group consisting of beeswax, candelilla wax, sunflower wax, carnauba wax, rice bran wax, orange peel wax, grapefruit wax, lemon peel wax, sunflower wax and sumac wax.

16. A skin or lip colorant of claim 1 which contains 1% to about 15 wt. % lanolin or lanolin derivative.

17. A skin or lip colorant of claim 1 which contains from about 20% to about 80 wt. % of a natural powder base.

18. A skin or lip colorant of claim 17 in which the powder base is selected from the group consisting of talc, corn starch, microcrystalline cellulose, micronized calcium carbonate, kaolin, mica and bentonite clay.

19. A lipstick of claim 1 comprising (1) from about 1% to about 15 wt. % micronized colored herbal extract powder in from about 4% to about 60 wt. % of a natural or naturally derived vegetable oil, (2) from about 0.5% to about 6 wt. % of a natural colorant mineral ore powder, (3) from about 2% to about 50 wt. % of a natural or naturally derived emollient liquid ester of a C₁ to a C₁₈ alcohol and a C₁₂ to a C₁₈ fatty acid or a natural or naturally derived ester of a C₁ to a C₁₈ alcohol with a poly-carboxylic acid, and (4) from about 5% to about 20 wt. % of a vegetable based anhydrous wax.

20. The use of a skin or lip colorant of claim 1 as a lipstick, lip gloss, lipstick colorant lotion, skin blush, eye shadow, blemish cover or product for coloring the skin.

21. A method of coloring skin or lip by applying a composition comprising a dispersion of from about 1% to about 15 wt. % by weight of a micronized colored herbal extract powder dispersed in from about 4% to about 60 wt. % of a natural or naturally derived vegetable oil and further comprising optionally one of more of the following: (1) from about 0.5% to about 6 wt. % a natural colorant mineral ore powder, (2) from 1% to about 10 wt. % of a natural or naturally derived thickening agent, (3) from about 2% to about 50 wt. % of a natural or naturally derived emollient liquid ester of a C₁ to a C₁₈ alcohol and a C₁₂ to a C₁₈ fatty acid, (4) from 1% to about 50 wt. % of a natural or naturally derived ester of a C₁ to a C₁₈ alcohol with a poly-carboxylic acid, (5) from about 5% to about 20 wt. % of a vegetable based anhydrous wax, (6) from about 1% to about 15 wt. % lanolin or lanolin derivative or (7) from about 20% to about 80 wt. % of a natural powder base.
